(19) United States
(12) Patent Application Publication
(10) Pub. No.: US 2025/0186430 A1
(43) Pub. Date: Jun. 12, 2025

(54) THERAPY OF INTEROCEPTION DISORDERS WITH CGRP RECEPTOR ANTAGONISTS

(71) Applicant: Alberto CHIARUGI, Firenze (IT)

(72) Inventor: Alberto CHIARUGI, Firenze (IT)

(21) Appl. No.: 18/837,127

(22) PCT Filed: Jan. 19, 2023
(86) PCT No.: PCT/IT2023/050017

§ 371 Date: Aug. 8, 2024

(30) Foreign Application Priority Data
Jan. 20, 2022 (IT) 102022000000869

(51) Int. Cl. A61K 31/496; A61K 31/4545; A61P 3/00; A61P 43/00

(52) U.S. Cl. CPC A61K 31/496; A61K 31/4545; A61P 3/00; A61P 43/00

(57) ABSTRACT

Interoception is the mechanism by which an individual is informed about the status of its own body. When interoception becomes dysfunctional, it turns into a deleterious event for the health status of an individual. The use of calcitonin gene-related peptide (CGRP) receptor antagonists (gepants) for the treatment and 70 prevention of interoception disorders is described.

### TECHNICAL FIELD

The present invention refers to the field of drugs for the treatment of disorders of interoception

### BACKGROUND ART

Interoception is the mechanism by which an individual is informed about the status of its own body. Interoception allows each individual to gather constant information of the functioning of internal organs so that, in case of dysfunction, specific responses can be activated to restore homeostasis. By so doing, interoception allows an individual to discriminate between the correct or deranged functioning of organs and functions (the health status) such as the gastrointestinal tract, the respiratory apparatus, appetite and satiety, the emotional status, sentiments, motivation as well as the presence of pain. In this perspective, perception of malaise results from interoceptive information signaling a negative health status. Interoception is therefore the opposite of exteroception that regulates the consciousness of the body in the environment.

From a neurophysiological point of view, the health status defined by interoceptive signaling is mediated by neuronal afferents belonging to both the sensory (mechanical, thermal, nociceptive) and autonomous nervous system. These peripheral afferents first project to brainstem nuclei (mostly the parabrachial nucleus) and are then redirected to the hypothalamus, amygdala, thalamus from which interoceptive information reaches the parietal, frontal and insular cortex where the consciousness and affective components of the health status are decoded.

It is well acknowledged that one of the most common homeostatic responses activated in an individual receiving a negative interoceptive information (signals of malaise) is appetite loss also known as anorexia. Even though such a response may appear paradoxical, it is, indeed, a key ancestral response in animals aimed at protecting the subject by preventing assumption of food that potentially may have caused the sickness condition. Of course, when the anorectic response is prolonged, it turns into a deleterious event for the health status, leading to excessive reduction of caloric intake, weight loss, weakness and a general derangement of the defenses of the organism. It is known that numerous hepatic, renal, gastrointestinal, immune, infective and painful disorders are invariantly associated with reduced appetite and anorexia. Likewise, a similar anorectic response is triggered by intoxication or pharmacological treatments. Among the latter, anticancer chemotherapy is very frequently associated with anorexia. Unfortunately, this type of anorexia frequently reaches such a severe intensity to critically reduce the patients' caloric intake. This, in turn, prompts therapy interruption that, inevitably, leads to chemotherapy failure and unrestricted tumor growth. It is also worth noting that the neoplastic patient is exposed to another risk of weight loss/anorexia, i.e. tumor cachexia, a serious clinical condition in which appetite loss is accompanied by loss of adipose and muscle tissues triggered by tumor-derived signals.

In light of the high frequency of food disorders related to negative interoceptive signaling and the ensuing severe clinical conditions, great effort has been directed at the identification of drugs able to restore nutritional homeostasis in these individuals.

The problem to be solved is therefore that of identifying drugs able to counteract interoceptive signaling during different types of disorders.

Even though neurochemistry of interoception is still in large part undeciphered, specific neuropeptides that modulate central processing of sensory afferents and signal malaise have been identified at the preclinical level. Neuropeptides are small (10-40 amino acids) proteinaceous molecules that play a myriad of neuronal and neuroendocrine functions within the central and peripheral nervous system. Of note, neuropeptides are released in a vesicle-dependent manner, but, at variance with classic small-molecule neurotransmitters such as glutamate, serotonin or noradrenaline, neuropeptides prompt long-lasting postsynaptic signaling that, typically, reaches neurons very distant from the site of release, thereby sustaining the so-called “volume transmission”.

Among the numerous neuropeptides, the calcitonin gene-related peptide (CGRP) is a 37 amino acid molecule with central and peripheral actions. Although the peripheral vasodilating effects of CGRP have been deeply investigated and characterized, the central effects of the neuropeptide in large part still wait to be identified. It is well known that such a lack of knowledge must be mainly ascribed to the inability of peripherally acting CGRP receptor agonists and antagonists to cross the blood-brain barrier. Such as impermeability inevitably hamper the possibility of using these tools to modulate CGRP neurotransmission and understand its role in neurophysiopathology (Nat Rev Neurol. 2018; 14:338-35). To circumvent such a technical problem, scientists have modulated CGRP 75 neurotransmission by means of intracerebral injection of drugs regulating the neuropeptide receptor or viruses expressing proteins regulating CGRP neurotransmission. Remarkably, this technique is not clinically suitable and exploitable.

Thanks to these invasive experimental procedures, a key role of CGRP in regulating emotional functions such as responses to stress and aversive stimuli has recently emerged. As far as the aversive stimuli are concerned, it is now known that CGRP is a pivotal player of interoceptive information that reaches the parabrachial nucleus and is then redirected to the amygdala. This signaling pathway seems to play an important role is integrating sensory and autonomic afferents carrying interoceptive information in order to prompt specific homeostatic responses to different types of stresses, pain and malaise in general (Trends Neurosci. 2018; 41:280-293). On this basis, it would be theoretically possible to modulate interoception by regulating CGRP neurotransmission within the central nervous system. Unfortunately, no drugs have been so far identified that, administered in the periphery adopting a clinically suitable and exploitable method, can reach the brain and regulate CGRP-dependent interoceptive signaling within the central nervous system.

Antagonists of the CGRP receptors, typically referred as “gepants”, have been developed for the symptomatic treatment of migraine. The first gepants such as olcegepant and telcagepant proved efficacious as anti-migraine drugs but their development was stopped because of the occurrence of hepatotoxicity. Recently, new, non-hepatotoxic gepants such as ubrogepant, rimegepant, atogepant and zavegepant have been developed for the symptomatic treatment of migraine. It is known that gepants do not penetrate within the brain because they are unable to cross the blood brain barrier (J. Pharmacol. Exp. Ther. 2013, 347:478-86). It is known, indeed, that gepants exert their anti-migraine effects at the level of the trigeminovascular afferents within the meninges (Nat. Rev. Neurol. 2018; 14:338-350; Cephalalgia. 2020; 40:924-934).

Hence, the expert of the filed does not find any clue in the state of the art that gepants act within the central nervous system. Likewise, the expert of the field does not find any clue in the state of the art that gepants regulate interoception, and the homeostatic responses prompted by cerebral processing of interoceptive information.

The technical problem to be solved, therefore, is that of identifying drugs able to inhibit the actions of CGRP within the brain to modulate CGRP-dependent interoceptive information and the responses prompted by interoceptive signaling that cause malaise and negatively impact the health status of an individual.

### Disclosure of the Invention

Unexpectedly, we have now found that the oral (i.e. peripheral) administration of gepants such as rimegepant, ubrogepant, atogepant and zavegepant, is able to modulate cerebral processing of interoceptive information (i.e. malaise) in numerous experimental models of human disorders.

Specifically, we have unexpectedly found that oral administration of rimegepant, ubrogepant, atogepant and zavegepant reduces food aversion/anorexia, body weight loss and accelerates weight body recovery in rats exposed to the chemotherapeutic drug cisplatin (6 mg/kg, intraperitoneal), as an experimental model of malaise due to antineoplastic chemotherapy (FIG. 1).

Further, we have unexpectedly found that oral administration of rimegepant, ubrogepant, atogepant and zavegepant reduces food aversion/anorexia, weight loss and increases spontaneous motility in mice with chronic arthritis due to injection of Freund's adjuvant as an experimental model of malaise due to chronic pain (FIG. 2).

In keeping with these findings, we have unexpectedly found that oral administration of rimegepant, ubrogepant, atogepant and zavegepant completely prevents weight loss and in part even food aversion/anorexia in rats subjected to laparotomy and gut manipulation as an experimental model of malaise due to abdominal surgery (FIG. 3).

Further, we have tested the gepants in a model of hepatopathy, a condition that typically prompts asthenia, weight loss and anhedonia. We have unexpectedly found that oral administration of rimegepant, ubrogepant, atogepant and zavegepant reduces weight loss and triggers an increase in sweet water consumption (the latter as an index of motivational behavior and reduced anhedonia) in rats treated with carbon tetrachloride as a model of malaise due to hepatopathy (FIG. 4).

Further, we have unexpectedly found that oral administration of rimegepant, ubrogepant, atogepant and zavegepant prevents weight loss and reduction in spontaneous motility in rats injected with cerulein as an experimental model of pancreatitis (FIG. 5).

Further, we have tested the gepants in rats treated with a diet containing 0.75% adenine as an experimental model of renal insufficiency. We have unexpectedly found that oral administration of rimegepant, ubrogepant, atogepant and zavegepant reduces weight loss and decrease of food consumption/anorexia in the animals (FIG. 6).

Further, we have unexpectedly found that oral administration of rimegepant, ubrogepant, atogepant and zavegepant in mice sensitized and re-challenged with ovalbumin as a model of asthma or chronic obstructive pulmonary disease reduces parameters of malaise such as spontaneous motility, weight loss and decrease of sweet water consumption (FIG. 7).

As additional models of malaise due to interoceptive information, we have adopted those of tumor cachexia and sepsis. FIG. 8 shows the unexpected ability of the oral administration of rimegepant, ubrogepant, atogepant and zavegepant in reducing weight loss, and counteracting both loss of muscle mass and reduction of food consumption/anorexia in rats injected with Yoshida hepatoma, as a classic model of malaise due to tumor cachexia.

Similarly, we have unexpectedly found that oral administration of rimegepant, ubrogepant, atogepant and zavegepant reduces weight loss and decrease of food consumption/anorexia in rats injected intraperitoneally with lipopolysaccharides from Salmonella Typhimurium as a classic model of malaise due to sepsis/systemic immune activation (FIG. 9).

According to the invention, the gepants can be formulated and administered via the oral, intravenous, intraarterial, intramuscular, endonasal, transdermal and subcutaneous routes for the treatment of interoception disorders. The amounts of gepants to be administered are those commonly adopted for this type of drugs, for instance 10-3000 mg adopting daily, weekly, or monthly administration.

### BRIEF DESCRIPTION OF DRAWINGS

FIG. 1. Effect of rimegepant, ubrogepant, atogepant and zavegepant (10 mg/kg orally, daily, starting two days before cisplatin) on food consumption/anorexia, weight loss and weight body recovery in rats exposed to cisplatin (6 mg/kg, intraperitoneal), as an experimental model of malaise due to antineoplastic chemotherapy. * p<0.05, ** p<0.01, *** p<0.001, vs control, ANOVA and Tukeys post hoc test.

FIG. 2. Effects of rimegepant, ubrogepant, atogepant and zavegepant (10 mg/kg orally, daily, starting two days before arthritis induction) on food consumption/anorexia, weight loss and spontaneous motility in mice with chronic arthritis due to injection of Freund's adjuvant as an experimental model of malaise due to chronic pain. * p<0.05, ** p<0.01, *** p<0.001 vs Arthritis, ANOVA and Tukeys post hoc test.

FIG. 3. Effects of rimegepant, ubrogepant, atogepant and zavegepant (10 mg/kg orally, daily, starting two days before surgery) on body weight loss and food consumption/anorexia in rats subjected to laparotomy and gut manipulation as an experimental model of malaise due to abdominal surgery. * p<0.05, ** p<0.01, *** p<0.001 vs Laparotomy, ANOVA and Tukeys post hoc test.

FIG. 4. Effects of rimegepant, ubrogepant, atogepant and zavegepant (10 mg/kg orally, daily, starting two days before carbon tetrachloride) on body weight loss and sweet water consumption in rats treated with carbon tetrachloride as a model of malaise due to hepatitis/hepatopathy. * p<0.05, ** p<0.01, *** p<0.001 vs Hepatitis, ANOVA and Tukeys post hoc test.

FIG. 5. Effects of rimegepant, ubrogepant, atogepant and zavegepant (10 mg/kg orally, daily, starting two days before cerulein treatment) on body weight loss and spontaneous motility in rats injected with cerulein as an experimental model of pancreatitis. * p<0.05, ** p<0.01, *** p<0.001 vs Pancreatitis, ANOVA and Tukeys post hoc test.

FIG. 6. Effects of rimegepant, ubrogepant, atogepant and zavegepant (10 mg/kg orally, daily, starting two days before exposure to the nephrotoxic diet) on body weight loss and food consumption/anorexia in rats exposed to a diet containing 0.75% adenine as an experimental model of renal insufficiency. *p<0.05, ** p<0.01, *** p<0.001 vs Kidney Failure, ANOVA and Tukeys post hoc test.

FIG. 7. Effects of rimegepant, ubrogepant, atogepant and zavegepant (10 mg/kg orally, daily, starting two days before antigen rechallenge) on spontaneous motility, body weight loss and sweet water ingestion of mice sensitized and re-challenged with ovalbumin as a model of asthma or chronic obstructive pulmonary disease. * p<0.05, ** p<0.01, *** p<0.001 vs Kidney Failure, ANOVA and Tukeys post hoc test.

FIG. 8. Effects of rimegepant, ubrogepant, atogepant and zavegepant (10 mg/kg orally, daily, starting two days before tumor injection) on body weight loss, muscle mass and food consumption/anorexia in rats injected with Yoshida hepatoma, as classic model of malaise due to tumor cachexia. * p<0.05, ** p<0.01, *** p<0.001. **** p<0.0001 vs Hepatoma, ANOVA and Tukeys post hoc test.

FIG. 9. Effects of rimegepant, ubrogepant, atogepant and zavegepant (10 mg/kg orally, daily, starting two days before lipopolysaccharides injection) on body weight loss and food consumption/anorexia in rats injected intraperitoneally with lipopolysaccharides from Salmonella Typhimurium as a classic model of malaise due to sepsis/systemic immune activation. * p<0.05, ** p<0.01, vs Sepsis, ANOVA and Tukeys post hoc test.

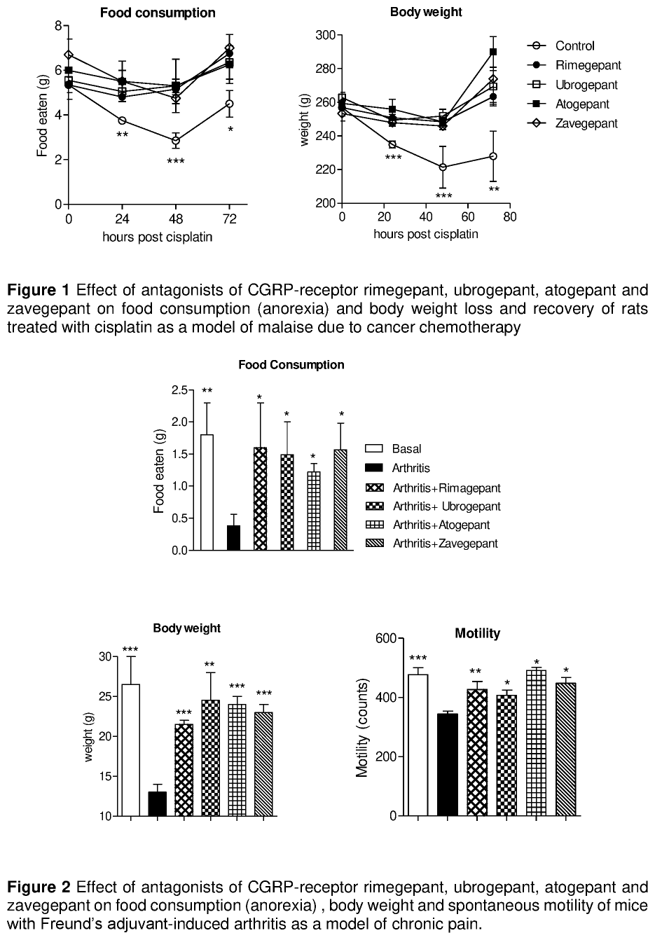

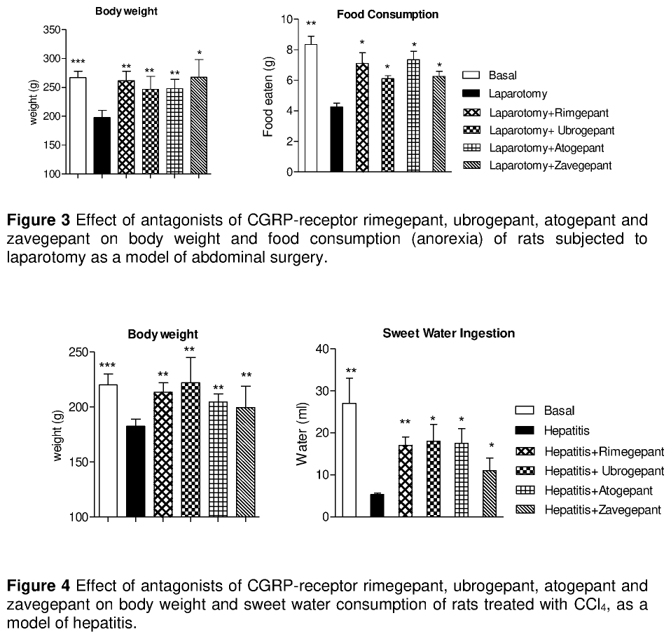

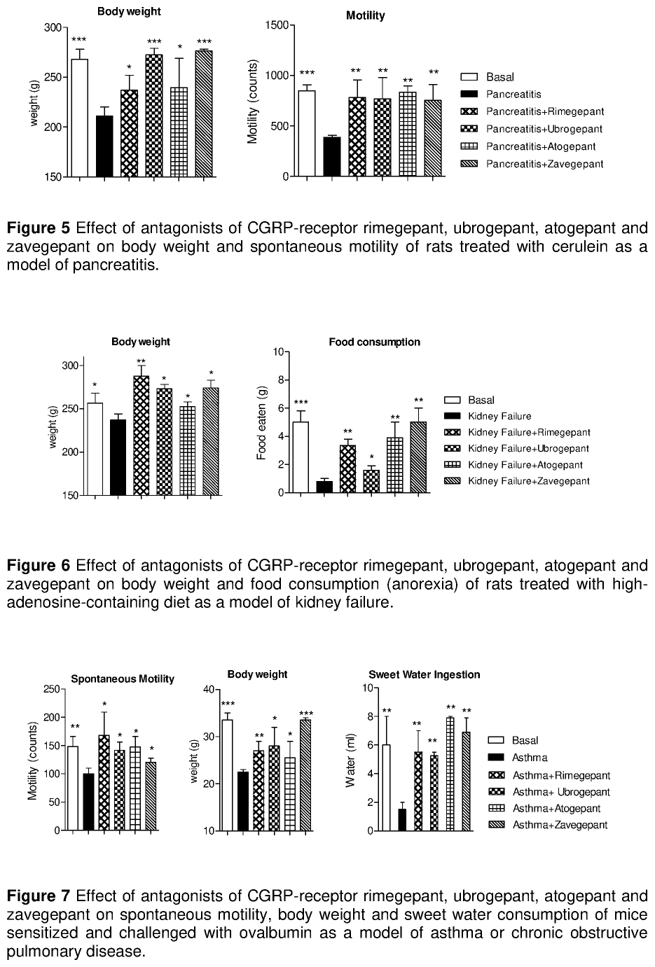

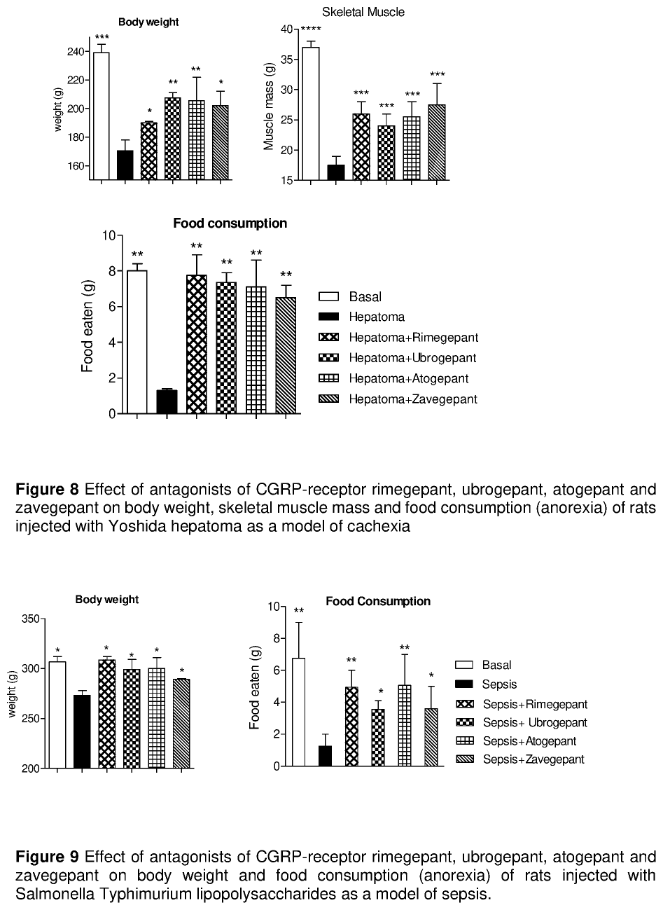

### BEST MODE FOR CARRYING OUT THE INVENTION

The best mode for carrying out the invention is to treat patients during interoceptive disorders or at risk of interoceptive disorders with daily, weekly, or monthly doses of rimegepant, ubrogepant, atogepant and zavegepant administered by different routes such as, for example, but not limited to, oral or intravenous.

1. Antagonists of CGRP receptor and pharmaceutically acceptable formulations thereof for the use in the prevention and therapy of malaise (negative perception of the health status in an individual).

2. Antagonists of CGRP receptor and pharmaceutically acceptable formulations thereof for the use in the prevention and therapy of eating disorders or body weight loss.

3. Antagonists of CGRP receptor and pharmaceutically acceptable formulations thereof for the use to stimulate appetite and body weight recovery.

4. Antagonist of CGRP receptor for use according to claims 1-3 in case said antagonist is Rimegepant.

5. Antagonist of CGRP receptor for use according to claims 1-3 in case said antagonist is Ubrogepant.

6. Antagonist of CGRP receptor for use according to claims 1-3 in case said antagonist is Atogepant.

7. Antagonist of CGRP receptor for use according to claims 1-3 in case said antagonist is Zavegepant.

8. Antagonists of CGRP receptor (gepants) for use according to claims 1-7 in an individual affected by neoplastic (cachexia), inflammatory, autoimmune, rheumatic, respiratory chronic obstructive and fibrotic, gastroenteric, hepatic, pancreatic, abdominal, renal, cardiologic, neurological, and psychiatric disorders.

9. Antagonists of CGRP receptor (gepants) for use according to claims 1-7 in an individual affected by chronic or postsurgical pain.

10. Antagonists of CGRP receptor (gepants) for use according to claims 1-7 in an individual exposed or to be exposed to anti-tumoral therapy (chemotherapy).
